(19) United States
(12) Patent Application Publication
(10) Pub. No.: US 2026/0224197 A1
(43) Pub. Date: Aug. 6, 2026

(54) DEVICES AND METHODS FOR FREEHAND MULTIMODALITY IMAGING

(71) Applicant: Trustees of Tufts College, Medford, MA (US)

(72) Inventors: Srivalleesha Mallidi, Medford, MA (US); Deeksha Sankepalle, Medford, MA (US)

(21) Appl. No.: 19/150,269

(22) PCT Filed: Jan. 23, 2024
(86) PCT No.: PCT/US2024/012599

§ 371 Date: Jul. 23, 2025

Related U.S. Application Data
(60) Provisional application No. 63/440,687, filed on Jan. 23, 2023.

(51) Int. Cl. A61B 8/00; A61B 5/00; A61B 34/20

(52) U.S. Cl. CPC A61B 8/4254; A61B 5/0095; A61B 8/4444; A61B 2034/2048; A61B 2034/2055

(57) ABSTRACT

An apparatus and method for integrated ultrasound and photoacoustic imaging (USPAI) and visual odometry (VO) three-dimensional (3D) USPA imaging reconstruction is described. The apparatus is configured for freehand scanning. A processor is configured to synchronize the imaging scans acquired by the USPA probe and odometer data from the at least one optical sensor and inertial measurement unit (IMU) of the visual odometer, whereby the data from their respective components is timestamped. Alignment and smoothing steps enable accurate 3D reconstruction of the two-dimensional UPSA imaging scans from the linear translational motion of the USPA probe.

### CROSS REFERENCE TO RELATED APPLICATIONS

The present application is based on, claims priority to, and incorporates herein by reference in its entirety for all purposes, U.S. Provisional Application Ser. No. 63/440,687, filed Jan. 23, 2023.

### STATEMENT OF GOVERNMENT SUPPORT

This invention was made with government support under grant number UL1TR002544 awarded by the National Institutes of Health. The government has certain rights in the invention.

### BACKGROUND

Ultrasound (US) imaging, specifically US B-mode imaging, enables clinicians and sonographers to view and evaluate tissue anatomy non-invasively. However, the orientation, volume or complex structure of the anatomy is difficult to visualize using just two-dimensional (2D) images. The need for reconstructed three-dimensional (3D) volumes, along with larger field of view is undebatable as it can aid clinicians to better visualize anatomy and function as a whole. Additionally, 3D volumes can help surgeons to ascertain whether a surgical instrument is placed accurately within the region of interest. Specifically if multi-parametric 3D information can be obtained from tissues, it will lead to better prognosis and/or monitoring of treatment efficacy. Particularly for applications in cancer theranostics and vascular malignancies, 3D combined US and photoacoustic imaging (USPAI) has the potential to substantially improve clinical outcomes by providing this multi-parametric information on tissue morphology and function.

There have been several advances recently in 3D US imaging. Given the advantages of combining US with the PAI modality, particularly 3D USPA imaging has not been exclusively studied up until recently due to several reasons listed below. First, 2D array transducers can be used to generate 3D images, however they are very expensive, limited for specific organs such as the ring-shaped array transducers used for breast imaging, or the systems are not portable. Second, mechanical translation of the transducer and optical fiber for USPA imaging is accomplished by attaching the integrated probe to a linear stage as been shown in several studies. For example, breast imaging by Nyayapathi et al., preclinical murine tumors imaged by Mallidi et al. with FujiFilm Vevo LAZR-X system or the handheld system proposed by Lee et al. use translational stages to obtain 3D images. Such translation stage-based systems have limited range of motion and are restricted by the range or length of the linear stages being used. Particularly for motion of transducer that is attached to a non-mobile stage, the clinical applications will be limited due to lack of flexibility. Third, fiducial markers such as tattoos have been used for 3D reconstruction of photoacoustic images. For example, Holzwarth et al. suggested an optical pattern be used as a global coordinate system, where a pre-set tattoo-like grids are placed on the region of interest. These high-contrast tattoo grids act as a guide for estimating the position of the transducer in each image. Although this study was able to achieve 3D reconstruction without any modifications to the transducer or imaging equipment, it requires the application of a tattoo grid on the area of interest before imaging, which is not conducive for several clinical applications such as imaging a wound site. Furthermore, there will be a limitation on the area that can be scanned using the technique along with requirement of extensive reconstruction methods for non-linear or curved surfaces. Fourth, 3D imaging was performed with application specific modulation of light delivery, transducer and customized reconstruction using various algorithms; however, they are computationally expensive, time-consuming and system specific methodologies. Lastly, mechanical localizers and robotic arms such as the da Vinci robot have been used for spatially localized USPA imaging. Such systems, though cost-efficient, have limited availability and can be bulky.

### SUMMARY

The present disclosure provides systems and methods for freehand USPA imaging and 3D reconstruction that overcome the aforementioned drawbacks using an visual odometer (VO) to track a USPA-capable probe in a 3D reference frame. An apparatus may include an integrated imaging probe and VO, where 2D USPA scans are synchronized with position data from the VO to reconstruct a 3D image to obtain combined structural and functional imaging of a sample.

In one aspect of the present disclosure, an apparatus for 3D image reconstruction is presented. The apparatus comprises a housing configured for freehand movement within a 3D reference frame defined by an X_{W}-axis, Y_{W}-axis, and Z_{W}-axis. The housing comprises a laser source configured to transmit an electromagnetic (EM) wave into a sample to produce a photoacoustic effect therein and an ultrasound probe configured to generate scans of the sample. The housing also includes a visual odometer configured to track movement of the ultrasound probe in the 3D reference frame to generate odometer data. The housing further comprises a processor in communication with the ultrasound probe and the visual odometer. The processor is configured to receive the scans and odometer data, wherein the scans and odometer data each include timestamps, synchronize the scans and odometer data, and construct a 3D image of the scans based on the synchronization.

In another aspect of the present disclosure, a method for 3D image reconstruction is described. The method comprises receiving, using a processor, photoacoustic ultrasound scans from an ultrasound probe within a housing configured for freehand movement within a 3D reference frame defined by an X_{W}-axis, Y_{W}-axis, and Z_{W}-axis. The method further comprises receiving, using the processor, odometer data from a visual odometer within the housing configured to track movement of the ultrasound probe in the 3D reference frame. The method further includes synchronizing the scans and the odometer data based on timestamps associated with each of the scans and the odometer data and constructing a 3D image of the scans based on the synchronization.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1A is a schematic of an apparatus for three-dimensional (3D) image reconstruction, according to the present disclosure.

FIG. 1B is a schematic of an apparatus for 3D image reconstruction, according to the present disclosure.

FIG. 2 a flowchart depicting the data processing involved in generating motion-compensated 3D reconstructed USPA images from the visual odometer data.

FIG. 3 is a flowchart depicting a method for 3D image reconstruction, according to the present disclosure.

FIG. 4A is a custom 3D-printed handheld probe housing an US transducer, optical fiber for laser light delivery and Intel® RealSense™ T265 camera. The camera and USPA image co-ordinates are represented as X_{C}, Y_{C} and Z_{C} and X_{U}, Y_{U} and Z_{U} respectively.

FIG. 4B is a T265 camera that has two fisheye imagers and an integrated IMU where X_{C} is long axis, Y_{C} is the short axis, and Z_{C} is the height of the camera.

FIG. 5A is a schematic of the transformation of camera co-ordinates and image co-ordinates to the world co-ordinates. Camera co-ordinates and USPA system co-ordinates are represented as X_{C}, Y_{C} and Z_{C}, and X_{U}, Y_{U} and Z_{U} respectively.

FIG. 5B is a schematic representation of a phantom with a 0.7 mm lead (black) in between two supporting beams (grey) used for USPA imaging. Black arrow depicts the forward-backward direction of the integrated probe motion and yellow dashed line is the scan length.

FIG. 5C is a schematic representation of a phantom with a 0.7 mm lead (black) in between two supporting beams (grey) used for USPA imaging. Black arrow depicts the left-right direction of the integrated probe motion and yellow dashed line is the scan length.

FIG. 5D is a schematic representation of a phantom with a 0.7 mm lead (black) in between two supporting beams (grey) used for USPA imaging. Black arrow depicts the up-down direction of the integrated probe motion and yellow dashed line is the scan length.

FIG. 5E is the translational pose data when the integrated probe is moved in the X_{C}, Y_{C} and Z_{C} direction respectively in the world frame.

FIG. 5F is the translational pose data when the integrated probe is moved in the X_{C}, Y_{C} and Z_{C} direction respectively in the world frame.

FIG. 5G is the translational pose data when the integrated probe is moved in the X_{C}, Y_{C} and Z_{C} direction respectively in the world frame.

FIG. 5H shows USPA images of the pencil lead (point source) acquired over time during forward and backward motion of the integrated probe.

FIG. 5I shows USPA images of the pencil lead (point source) acquired over time during left and right motion of the integrated probe.

FIG. 5J shows USPA images of the pencil lead (point source) acquired over time during up and down motion of the integrated probe.

FIG. 5K is 2D PA and US frames acquired at the positions specified with orange and magenta arrows in FIG. 5E.

FIG. 5L is 2D PA and US frames acquired at the positions specified with the orange arrow in FIG. 5F.

FIG. 5M is 2D PA and US frames acquired at the positions specified with the orange arrow in FIG. 5G.

FIG. 5N is 2D PA and US frames acquired at the positions specified with the magenta arrow in FIG. 5E.

FIG. 5O is 2D PA and US frames acquired at the positions specified with the magenta arrow in FIG. 5F.

FIG. 5P is 2D PA and US frames acquired at the positions specified with the magenta arrow in FIG. 5G.

FIG. 5Q is a 3D reconstruction of 2D USPA images when camera is moved along the X_{C}, Y_{C} and Z_{C} axes respectively.

FIG. 5R is a 3D reconstruction of 2D USPA images when camera is moved along the X_{C}, Y_{C} and Z_{C} axes respectively.

FIG. 5S is a 3D reconstruction of 2D USPA images when camera is moved along the X_{C}, Y_{C} and Z_{C} axes respectively.

FIG. 6 is a plot of the accuracy of the camera pose data for various distance travelled (10-300 mm range) along different axis.

FIG. 7 is a plot of various step-sizes recorded on the T265 camera when mounted on to a linear stage with movement in the Y_{C} axis. The pose data shown represents the smoothened data for each step size after application of Savitsky-Golay filter.

FIG. 8A is a plot of raw pose data acquired for data presented in FIG. 7. Raw pose data is shown in blue line, Savitzky-Golay smoothened pose data overlaid as red line and the number of steps identified by a custom MATLAB code with the findpeaks command are shown as black triangular markers. Total of 67 steps for 150 μm step-size over a range of 10 mm.

FIG. 8B is a plot of raw pose data acquired for data presented in FIG. 7. Raw pose data is shown in blue line, Savitzky-Golay smoothened pose data overlaid as red line and the number of steps identified by a custom MATLAB code with the findpeaks command are shown as black triangular markers. Total of 34 steps for 300 μm step-size over a range of 100 mm.

FIG. 8C is a plot of raw pose data acquired for data presented in FIG. 7. Raw pose data is shown in blue line, Savitzky-Golay smoothened pose data overlaid as red line and the number of steps identified by a custom MATLAB code with the findpeaks command are shown as black triangular markers. Total of 62 steps for 500 μm stepsize over a range of 300 mm.

FIG. 8D is a plot of raw pose data acquired for data presented in FIG. 7. Raw pose data is shown in blue line, Savitzky-Golay smoothened pose data overlaid as red line and the number of steps identified by a custom MATLAB code with the findpeaks command are shown as black triangular markers. Total of 30 steps for 1000 μm step-size over a range of 300 mm.

FIG. 9A is a plot of the distance travelled by the integrated probe for various speeds programmed on the linear stage. The slope of the pose data provides the speed recorded by the camera.

FIG. 9B is a plot of the comparison of the speed on the linear stage to the speed recorded by the T265 camera (R2=0.986).

FIG. 9C shows images of motion compensated 3D reconstructed PA images for various speeds ranging from 1 to 10 mm/s. Inset: Photograph of the hair phantom (scale bar: 5 mm).

FIG. 10A is an image of a Tissue mimicking phantom with hair placed as ‘X’ in gelatin was used for freehand USPA scan (scale bar: 5 mm).

FIG. 10B is a plot of the speed measured by the T265 camera for users performing a freehand USPA scans. Average speed of each user is represented by the horizontal line.

FIG. 10C is a 3D reconstruction of motion compensated USPA scans along the X_{W} axis when imaged by a user at a lower speed (User 2: ~3.5 mm/s).

FIG. 10D is a 3D reconstruction of motion compensated USPA scans along the X_{W} axis when imaged by a user at a higher speed (User 3: 14 mm/s)

FIG. 10E is a plot of the corresponding translational pose data acquired by the T265 camera for the handheld scan shown in FIG. 10C.

FIG. 10F is a plot of the corresponding translational pose data acquired by the T265 camera for the handheld scan shown in FIG. 10D.

FIG. 11A is photograph of 3D printed blood vessel phantom.

FIG. 11B is 3D reconstructed PA and US images of the phantom (imaged area highlighted in blue rectangle; ~155 mm) using the handheld integrated probe described herein. PA and US images were acquired simultaneously using Vevo LAZR-X system. Scale bar: 10 mm.

FIG. 11C is a 3D reconstruction of blood vessel phantom (as shown in FIG. 11A) using a linear motor. The maximum scan length achievable on this motor was 45 mm (highlighted in yellow rectangle).

FIG. 11D is a plot of the translational pose data of the corresponding handheld scan.

FIG. 12A is a photograph of the rat spleen (ex vivo) embedded in 8% gelatin for handheld and motorized USPA imaging.

FIG. 12B is a plot of the translational pose data acquired by the T265 camera for a handheld scan shown.

FIG. 12C is a plot of the comparison of volumes estimated from the uncompensated and compensated handheld scans with the ground truth obtained from the linear motor scan. Each symbol in the graph represents a different user conducting the handheld scan (6 users). Percentage difference in volume was computed for all the 3D reconstructed spleen images. A significant difference (p<0.0001) in volume was observed between uncompensated and compensated groups.

FIG. 12D shows 3D US and PA images of ex vivo spleen in top (left column), side (center column) and front (right column) view for motorized scan (considered as ground truth) (top row) and handheld uncompensated scan (center row) and compensated scan (bottom row). Scale bar: 5 mm. Coordinates represent X_{C} (blue), Y_{C} (red) and Z_{C} (green).

### DETAILED DESCRIPTION

There is an increasing need for 3D ultrasound and photoacoustic (USPA) imaging technology for real-time monitoring of dynamic changes in vasculature or molecular markers in various malignancies. Current 3D USPA systems utilize expensive 3D transducer arrays, mechanical arms or limited-range linear stages to reconstruct the 3D volume of the object being imaged. Overall, there is a need for handheld USPA system, that can be low cost, portable, attachable to any transducer and light delivery system (i.e., be system independent), not limited in range of motion and conducive for both linear and rotational translation (i.e., have six degrees of freedom of movement in 3D space).

Described herein are systems and method directed to an economical, portable, and clinically translatable handheld device for 3D USPA imaging. In a non-limiting example, the systems and methods may use of an off-the-shelf and low-cost visual odometry system for freehand 3D USPA imaging that can be seamlessly integrated into several photoacoustic imaging systems for various clinical applications.

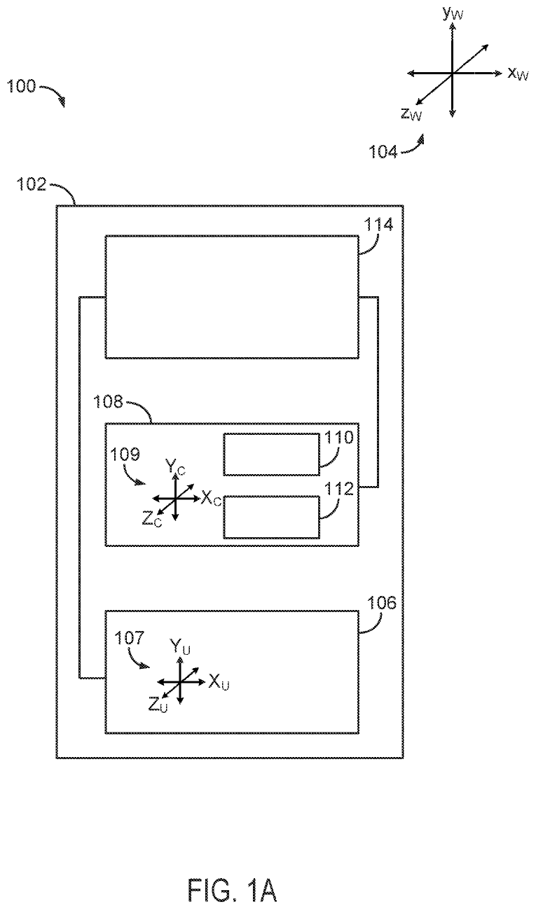

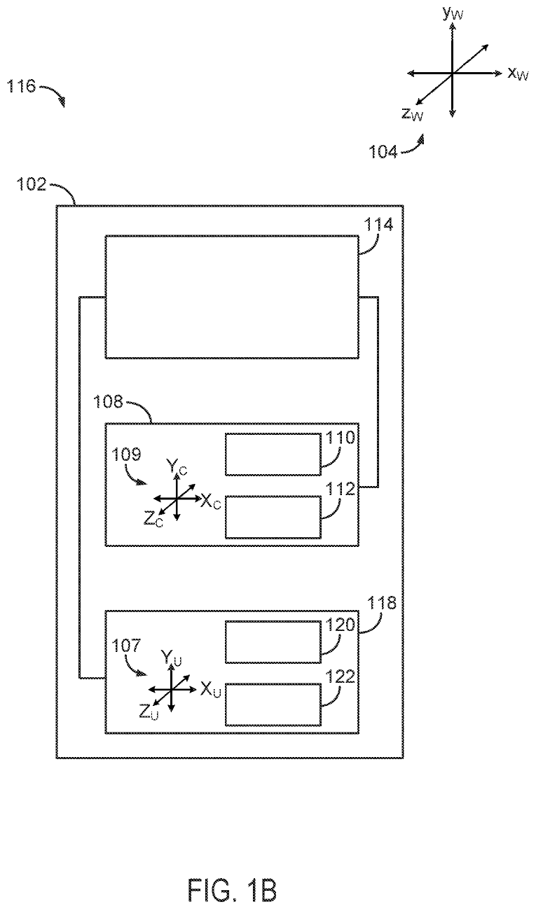

FIG. 1A illustrates an apparatus 100 for freehand imaging of a sample. The apparatus 100 includes a housing 102 that is configured to be gripped by a user's hand or a robotic arm for unrestricted motion with a 3D reference frame (“world frame”) 104 with X_{W}, Y_{W}, and Z_{W} coordinate axes. The housing includes an imaging probe 106. In a non-limiting example, the imaging probe is one of a 2D imaging modality such as, but not limited to, ultrasound, photoacoustic imaging (FIG. 1B), or optical coherence tomography (OCT). The imaging probe 106 generates 2D scans of the sample in X_{U}, Y_{U}, and Z_{U} co-ordinates 107.

The housing 102 further includes a visual odometer (VO) 108 and generates odometer data in X_{C}, Y_{C}, and Z_{C} co-ordinates 109. In a non-limiting example, the VO includes at least one optical sensor 110. The at least one optical sensor 110 may include monocular, monocular omnidirectional, stereo, stereo omnidirectional, or RGB-D cameras. In a non-limiting example, the at least on optical sensor includes, but it not limited to, a visible light camera, an infrared camera, ultraviolet light camera, or light detecting and ranging (LiDAR) camera. In another non-limiting example, the at least one optical sensor includes a fisheye camera. In a non-limiting example, where multiple optical sensors are implemented, the camera types listed above may be mixed and matched. Further, the VO 108 includes an inertial measurement unit (IMU) 112. In a non-limiting example, the IMU includes a tri-axial gyroscope, tri-axial accelerometer, and optionally a tri-axial magnetometer.

As will be described in further detail in the Example below, an Intel® RealSense™ camera T265 may be used as the VO in the apparatus 100. For example, the camera includes a 6-axis IMU and two fisheye cameras and utilizes integrated simultaneous localization and mapping (SLAM) based on chip processing to determine pose and orientation odometer data. Alternatively, a Luxonis Oak-D VO may be used which includes a 9-axis IMU and two RGB cameras. However, this VO does not include integrated SLAM processing and requires custom SLAM algorithm development to determine pose and orientation odometer data. Another VO includes the ZED 2 Stereo camera including a 6-axis IMU and two cameras. The ZED uses SLAM based on chip processing to determine pose and orientation odometer data. Another example includes the MYNT eye camera with a 6-axis IMU and two cameras.

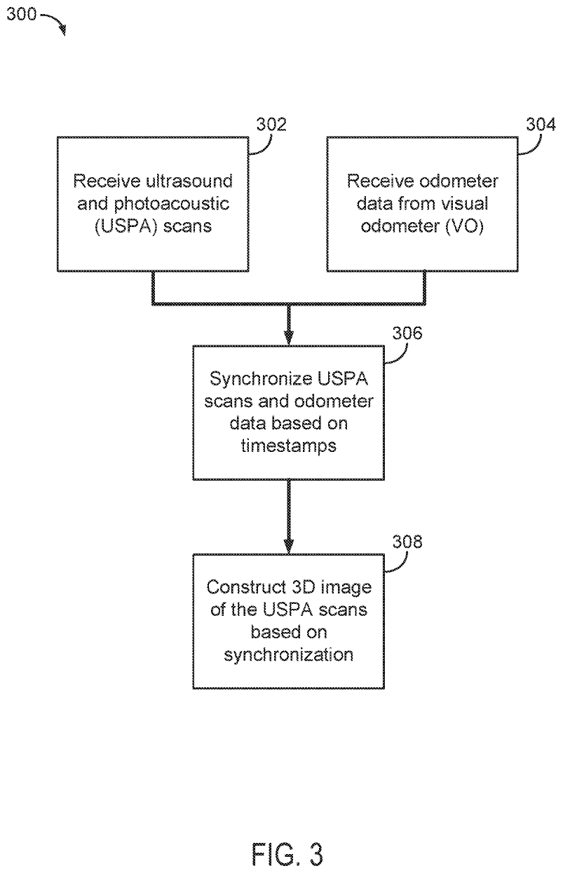

The housing 102 further includes a processor 114 in communication with the imaging probe 106 and VO 108. Alternatively, all or a portion of the processor may be external to the housing 102 and connect to the imaging probe 106 and VO 108 via wired or wireless connection. The functions of the processor are described in further detail below with respect to FIG. 3.

In a non-limiting example, the processor 114 is configured to collect image data of an environment (for example, an examining room) in which the sample is examined using the apparatus 100. The processor 114 may be configured to process the image data acquired by the at least one optical sensor 110 to identify one or more landmarks. These landmarks may include visually well-defined points, edges or corners of surfaces, fixtures, and/or objects in the imaging environment. For example, the at least one optical sensor 110 may be directed away from the sample, such as toward the ceiling of an examination room to identify existing features or purposely-placed markers. In another example, the at least one optical sensor is directed towards the sample and the processor 114 identifies some surface feature or landmark of the sample such as one or more anatomical features of the sample, the anatomical features including one or more of tissue surfaces, tissue boundaries or image texture of ordinary anatomical or pathological structures of the sample.

In a non-limiting example, the processor is further configured to calculate, in real time, the probe's X, Y and Z location as well as the probe's pitch, yaw, and roll orientation with respect to these landmarks.

The processor 114 may also be communicatively coupled with at least one IMU 112. The IMU 112 may be configured to measure translational and rotational motion of the apparatus 100. Using VO techniques, the processor 114 may be configured to estimate, in real-time, the probe's spatial position. Alternatively, or in addition, SLAM techniques and image registration techniques may be used. As a result, the combination of optical sensor data and IMU data will enable a reasonably accurate estimation of the probe's spatial position. Thus, the estimation of the probe's position may be based on a combination of data from the IMU 112 and the at least one optical sensor 110.

In a non-limiting example, the processor 114 may be configured to receive odometer data from the inertial sensor 112 and the at least optical sensor 110 and imaging scans from the imaging probe 106, and to use the received odometer data and imaging probe scans to determine the spatial position of the apparatus 100. For example, the processor may be configured to estimate a 6-DOF spatial position of the apparatus 100 using a combination of outputs from the imaging probe 106, the IMU 112 and the at least one optical sensor 110.

In a non-limiting example, the processor 114 may be further configured to process imaging probe scans using the determined spatial position of the apparatus 100. For example, a series of sequential 2D image scans may be collated to form a 3D image, after adjustment of each 2D image in view of the respective spatial position of the apparatus 100 at the time of obtaining each respective 2D image. As a result, imaging probe scans may be processed relative to the determined spatial position of the imaging probe 106, to determine the relative position, in 3D space, of each of a sequence of 2D scans. In a non-limiting example, the processor 114 further performs a transformation between the different systems (i.e., between the real world, VO, and imaging probe frame axes) to enable accurate 3D reconstruction is described in detail in the Example section below.

In a non-limiting example, the at least one optical sensor 110, IMU 112 and processor 114 may form part of an integrated unit. In an alternative embodiment of the apparatus 100, an optical sensor 110, IMU 112, and processor 114 may be integrated with the imaging probe 106 via appropriate attachment means in lieu of a housing 102. For example, the imaging probe may be configured to be gripped by the hand of a user to move the imaging probe 106, VO 108 and processor 114.

Referring now to FIG. 1B, an alternative apparatus 116 configured for 3D image reconstruction of USPAI is provided. Many of the structures and processes of FIG. 1A are identical to those in FIG. 1A. Here, the imaging probe is a USPA probe 118. The USPA probe 118 includes an ultrasound probe with one or more transducers 120 configured to acquire ultrasound scan data. The ultrasound data may comprise any ultrasound data type, such as B-mode ultrasound data, M-mode ultrasound data and Doppler ultrasound data, for example color Doppler ultrasound data. In a non-limiting example, the transducer 120 may be one-dimensional (1D) or 2D array transducers.

The USPA probe 118 further includes a laser source 122 configured to transmit an electromagnetic (EM) wave into the sample to produce a photoacoustic effect therein. In a non-limiting example, the laser source 122 includes, but is not limited to, one or more optical fibers connected to a laser system or light emitting diodes (LEDs). The optical wavelengths may be in the visible light and near infrared (NIR) range (200-2600 nm). In one example, the NIR spectral range (650-2500 nm) provides the greatest penetration depth into a sample of several centimeters. In a non-limiting example, where the laser source emits radiation in the NIR range and the at least one optical sensor 110 is an IR camera, the camera is preferably directed towards the ceiling to avoid signal interference between the laser and the camera.

When the sample is irradiated with the EM wave, the radiation is absorbed by specific tissue chromophores such as hemoglobin, melanin, water, lipids, or any contrast agent causing local heating and thermoelastic expansion. The thermoelastic expansion results in the emission of broadband, low-amplitude acoustic waves which may be detected at the surface of the sample by one or more ultrasound transducers. A resulting co-registered ultrasound and photoacoustic image of the sample may be formed by the processor 114 to provide functional and structural information.

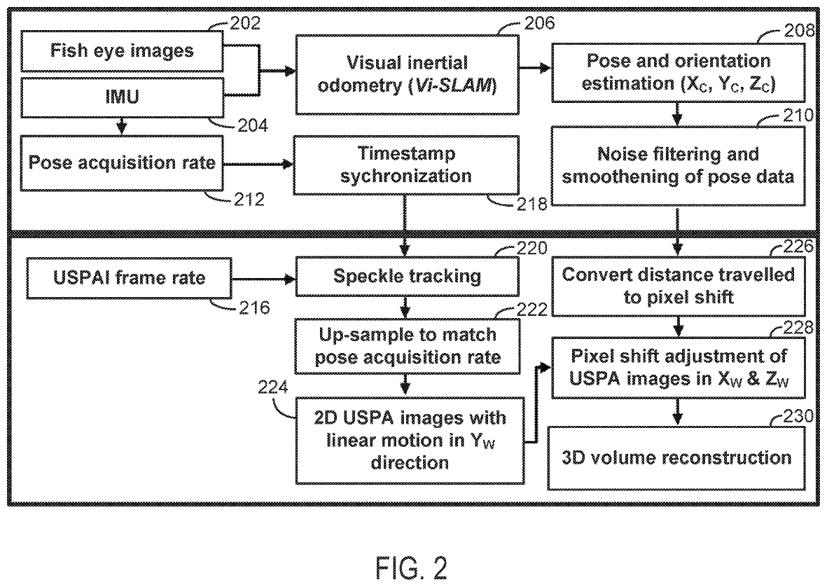

Referring now to FIG. 2, a detailed schematic of the processing steps of the processor 114 of FIG. 1B is shown. At step 202 and 204, odometer data from the at least one optical sensor 110 (e.g., two fisheye cameras) and/or IMU 112 is received by the processor 114, respectively. At step 206, the odometer data from the optical sensor 202 which comprises optical imaging data is fused with accelerometer, gyroscope, and optional magnetometer data from the IMU 112 using a visual inertial odometry (Vi-SLAM) algorithm. The resulting output data includes a 6-DOF pose and orientation estimation of the optical sensor in the VO frame of refence (X_{C}, Y_{C}, Z_{C}) at step 208. At step 210, the processor 114 is configured to apply a smoothing filter to the output data to generate smooth data with reduced noise.

At step 212, the processor acquires the pose acquisition rate of the odometer data from the at least one optical sensor 210 and IMU 112. The odometer data is timestamped.

At step 214, the processor receives timestamped ultrasound scan data from the USPA probe 118 to determine a USPA imaging frame rate. At steps 218, the pose data from the optical sensor and the USPA scans are synchronized based using the timestamps available on the data. In a non-limiting example, the USPA imaging and VO tracking are started simultaneously and/or synchronized via an external timer. Furthermore, the processor 114 is configured to identify a linear translational motion of a feature in the scans, such changes in speckle pattern to account for mismatches between the USPA probe 118 and the VO 108. At step 220, the synchronization obtained with timestamps is reconfirmed by the processor 114 by identifying motion based on the speckle pattern change in the ultrasound scans.

At step 222, the processor 114 is further configured to up-sample or interpolate the USPA scans to match the frame rate of the at least one optical sensor 110. At step 224 the processor determines the linear motion in the Y_{W} direction of the 2D USPA scans.

At step 226, the processor is further configured to convert the distance travelled by the at least one optical sensor 110 to a pixel shift by dividing the smoothed output data by a spatial resolution of the USPA probe in each of the X-axis, Y-axis, and Z-axis.

At step 228, the processor 114 spatially aligns the USPA scans in a 3D space relative to the X_{W} and Z_{W} directions based on the pixel shift determined in step 226 to reconstruct a 3D volume (step 230).

In accordance with another aspect of the disclosure, a method for 3D image reconstruction is provided. In a non-limiting example, any of the embodiments of the apparatus described previously may be utilized to perform the method. A non-limiting example of a general method 300 is shown in FIG. 3, while FIG. 2 and the Example section provide further detailed methods steps.

At step 302, a processor, such as processor 114, receives USPA scans. The processor further receives odometer data from the VO at step 304. In a non-limiting example, the USPA and odometer data each include timestamps. At step 306, the processor synchronizes the USPA scans and odometer data based on their timestamps and constructs a 3D image of the USPA scans based on the synchronization.

The following example provides additional non-limiting details pertaining to the apparatus and methods for 3D image reconstruction presented above, as well as example implementations and performance.

### Example

### 1. Introduction

Photoacoustic imaging (PAI) is a rapidly developing non-invasive imaging modality whose contrast depends on the tissue optical absorption properties. PAI has been employed in a wide range of applications from cancer to cardiovascular imaging. PAI takes advantage of the photoacoustic effect, in which absorbed photon energy from a pulsed light source produces a rapid thermoelastic expansion and contraction leading to generation of acoustic waves in tissues. The generated photoacoustic signals can be detected by an ultrasound (US) transducer and can be transformed into functional and molecular maps of tissue such as the tumor oxygen saturation or biomarker expression. Along with the ubiquitously available non-ionizing and non-invasive US imaging, PAI is now poised to join the armory of clinical imaging modalities. As US and PAI share similar receiver electronics, they can also be integrated into a single imaging system termed as “Ultrasound and Photoacoustic (USPA)” imaging as has been demonstrated previously by several groups.

A low-cost 3D USPA imaging system that has all the aforementioned salient features is described and characterized, namely portability, system independence, unlimited scanning range with six degrees of freedom of movement and low cost (<$$300). Specifically, a commercially available USPA transducer was coupled with the low-cost, commercially available T265 camera to obtain a portable, freehand 3D USPA imaging probe that can track freehand movements for 3D reconstruction without the use of fiducial markers. The compact size of the T265 camera (108×24.5×12.5 mm) and its lightweight nature (55 g) enable us to design an economical clinically translatable handheld 3D USPA imaging probe. The T265 camera consists of an Inertial Measurement Unit (IMU) and two fisheye cameras. A typical IMU unit consists of a tri-axial accelerometer, gyroscope, and sometimes a magnetometer. Algorithms like the Madgwick filter can fuse all three readings to compute a single orientation parameter called a quaternion. Integrating visual data from the fisheye camera using algorithms like Vi-SLAM (visual simultaneous localization and mapping) can further reliably provide information on the true position and linear velocity of the T265 camera. For example, Hausamann et al. used T265 camera to study the natural head motion of a subject while doing simple tasks such as walking, running, jog. In another study, Benjamin et al. utilized a similar sensor to capture the location of the US transducer to estimate the renal volume during a freehand 3D ultrasound scan of a kidney. Here for the first time, the utility of the RealSense camera to obtain 3D USPA images is investigated where handheld 2D images can be reconstructed into 3D volume from the quaternion information.

### 2. Methods and Materials

### 2.1 Phantom Fabrication

To characterize the imaging system and validate the reconstruction algorithm, several phantoms were utilized. The first phantom was fabricated by fixating a 0.7 mm diameter graphite pencil lead (Pentel, Hi-Polymer super 50HB) in between 3D printed supporting beams inside a box. This box was then filled with water for USPA imaging. The second phantom was made with two hair strands that were ~103 μm in diameter and placed in a ‘X’ (crisscross) configuration inside a custom 3D printed box filled with water. The phantom was used for characterizing the system for imaging speed, imaging range and resolution. To facilitate handheld imaging, a third phantom was fabricated with two hair samples in a crisscross configuration embedded in gelatin (CAS #9000-70-8, Sigma-Aldrich, St. Louis, Missouri). Briefly, gelatin powder (8% of w/v) was added to boiling water and stirred until the solution was clear. After the gelatin solution reached ~35° C., it was poured into the mold with the hair sample. The final gelatin block had dimensions 11.5 cm×8 cm×2.5 cm.

A fourth phantom was fabricated using a SCRIBD 3D stereo advanced drawing pen loaded with Polylactic acid filament (red color) to compare the range of motion of a linear stage to the integrated handheld probe. A blood vessel structure similar to that in a human arm was 3D printed and then embedded in an 8% w/v gelatin mold. Finally, a fifth phantom was fabricated with rat spleen in 8% w/v gelatin mold to quantify volume from the reconstructed 3D USPA images. As the focus of the optic fiber was at 10 mm, the spleen was positioned 10 mm deep in the gelatin phantom.

### 2.2 Ultrasound and Photoacoustic Imaging System

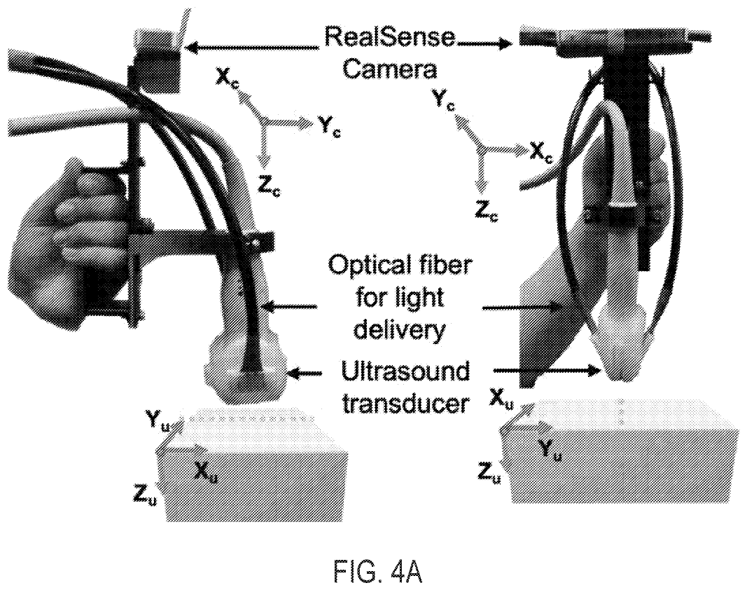

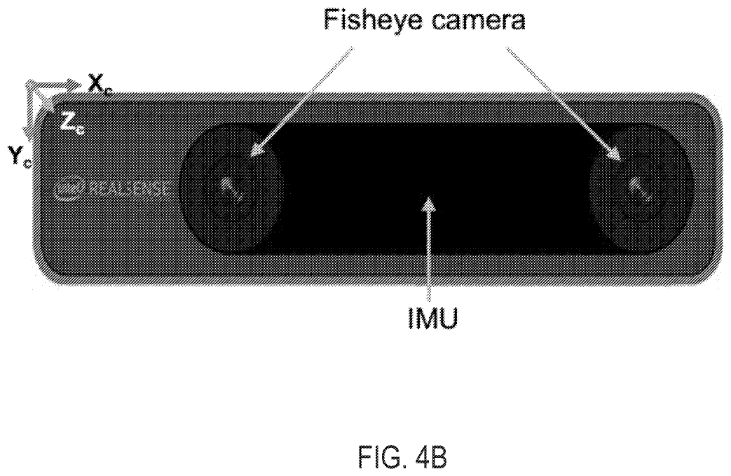

Vevo LAZR-X, a multimodality imaging system by VisualSonics (FUJIFILM, Ontario, Canada) with a 21 MHz transducer (MX250S) fitted with optical fiber jacket was used to acquire USPA images. The Vevo LAZR-X system is equipped with a 20 Hz tunable nanosecond pulsed laser. A default illumination wavelength of 750 nm was used for all experiments in this study as the laser had maximal energy output at this wavelength. The fibers focused light at 10 mm from the base of the transducer and hence all regions of interest in the phantoms were positioned to be 10 mm away from the transducer. Unless otherwise mentioned, USPA image acquisition was performed with no persistence, i.e., 5 Hz frame rate. A lightweight 3D printed mount was designed to hold the transducer, optical fibers, and the T265 camera (FIGS. 4A-4B). Together these parts will be referred as the “integrated” probe in the manuscript. The integrated probe also has a handle to enable users to comfortably hold it during the handheld scanning procedure as shown in FIG. 4A.

### 2.3 RealSense Hardware

The Intel® RealSense™ T265 camera consists of an IMU sensor (3 Degree of freedom, DOF gyroscope 2000°s range; 200 Hz sampling rate), and 3 DOF accelerometer (+4 g range; 62.5 Hz sampling rate) and 2 fisheye world cameras (173-degree diagonal field of view, 848×800-pixel resolution; 30 Hz sampling rate), which feed into a Vi-SLAM pipeline (FIG. 2). This algorithm fuses accelerometer, gyroscope, and wide-field image data into a 6 DOF estimation of position and orientation of the T265 camera relative to the environment. The data is computed on an onboard dedicated chipset in real-time which is proprietary to Intel Inc.

### 2.4 Imaging and Data Processing

The entire data acquisition and image processing flow is represented as a schematic in FIG. 2. The required software packages and wrappers, namely the Intel® RealSense™ SDK (Software Development Kit) and MATLAB wrappers were downloaded from GitHub (GitHub, CA). The Intel® RealSense™ data (.bag files) was recorded on the SDK application provided by Intel. All data and image processing were performed on MATLAB (MathWorks, Natwick, MA). USPA image data from VevoLab was imported into MATLAB for 3D reconstruction. The 3D volumes were then visualized in AMIRA (Thermo Fisher Scientific, Waltham, MA). The pose data from the camera and the USPA images were synchronized based using the timestamps available on the data. The synchronization obtained with timestamps was reconfirmed by identifying motion based on the speckle change in ultrasound images. In static conditions, the US images do not show changes in speckle pattern inside the region of interest in the phantoms. The time of scan and synchronized pose data was obtained from the start and end frames determined by start and end of the speckle change in phantoms.

### 2.5 Image Reconstruction Algorithm

Translational pose and camera frame acquisition rate were extracted from the Intel® RealSense™ SDK. The Intel® RealSense™ SDK uses Vi-SLAM to estimate the translational pose and orientation from fisheye images and IMU sensor data. Using ROS (Robotic Operating System) wrappers in MATLAB, the translational pose was imported onto MATLAB. Simultaneously, corresponding original USPA scan data set was imported into MATLAB. The pose data that contained relevant time stamps was then trimmed to match the USPA acquisition time. A smoothing filter (sgolay, degree of polynomial=0.01) was applied to eliminate jitter noise from the T265 camera. The smoothed pose data was divided by the spatial resolution of the transducer (calculated using Thorlabs NBS 1952) in each axis individually to determine the pixel shift. USPA scans were interpolated to match the frame rate of the T265 camera. Each frame from these scans were then spatially aligned in 3D space, based on the pixel shift previously determined. For visualizing the 3D structures, the voxel sizes were adjusted based on spatial resolution of the transducer.

### 2.6 Methodology to Evaluate Minimum Detectable Distance, Accuracy and Repeatability by the T256 Camera

A linear stage integrated with the FujiFILM Vevo LAZR-X system was used to obtain 3D images that acted as ground truth. To establish the least movement that can be reliably detected by the T265 camera setup, a linear scan of various step-sizes (150, 300, 500, 1000, 1500 and 2000 μm) for a travel range of 3 cm or 10 cm was performed on the hair phantom using the integrated probe. At every step, the persistence (number of frame averages) was set to “Max” to allow the system to average 20 USPA image frames (move the given step size distance, stop, acquire USPA imaging data and continue to move onto the next position. The number of imaging frames acquired were then compared to the number of steps (move-stop-move) detected from the pose data using findpeaks command in MATLAB. This experiment was repeated 3-5 times and the data was used to calculate the accuracy and repeatability of the steps and total distance moved by the camera.

### 2.7 Methodology to Characterize the Maximum User Speed Optimal for 3D Reconstruction

To characterize the maximum user speeds optimal for acceptable 3D reconstruction, the transducer was connected to a linear stage (X-LSM, Zaber, Vancouver, Canada) via custom 3D printed holder, to image the hair phantom at varying speeds of 0.5, 1, 2, 3.5, 5 and 10 mm/sec. USPA images were acquired continuously with no persistence. To synchronize the USPA imaging and the T265 camera data acquisition, recording on the T265 camera was started prior to acquiring USPA images. Furthermore, linear movement on the 3D X-Y-Z linear stage or handheld scan were initiated after a few baseline (no-motion) frames were acquired on the Vevo LAZR-X system.

### 2.8 User Evaluation of the Integrated Probe

Our next step was to evaluate handheld scans of the rat spleen phantom by six different users with previous experience on USPA imaging, particularly Vevo LAZR-X. During the handheld scan, the users were instructed to move the integrated probe with a constant speed to the best of their abilities. The users were able to watch the USPA images on the Vevo LAZR-X screen while scanning analogous to the clinical imaging scenario. Each user performed scan on the same phantom 3-5 times.

In this study, 3D reconstructed volume of a rat spleen was compared to further establish the performance of the 3D handheld design. A linear translational stage was used to scan the spleen phantom to obtain the 3D USPA images that was used to establish the ground-truth volume. The volume calculated from the handheld imaging by various users, i.e., the 3D images obtained after compensation due to the handheld motion detected by the T265 camera, was compared to the ground truth volume.

### 3. Results and Discussion

### 3.1 Relationship Between the T265 Camera and USPA Frame Axes

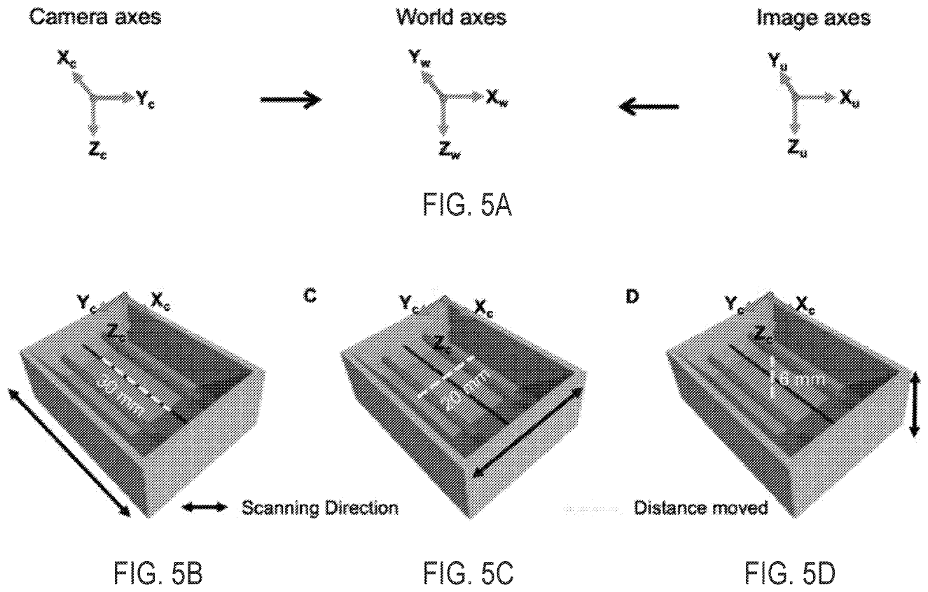

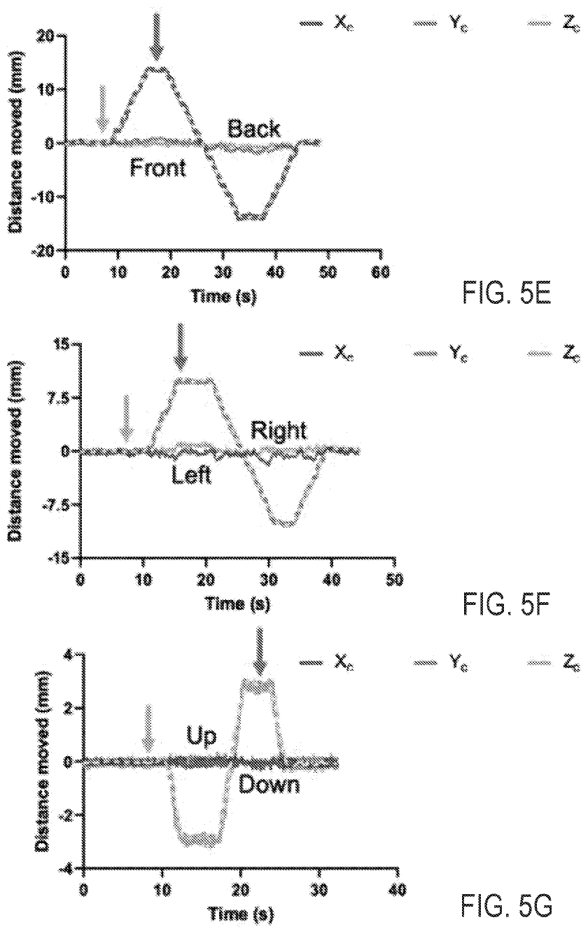

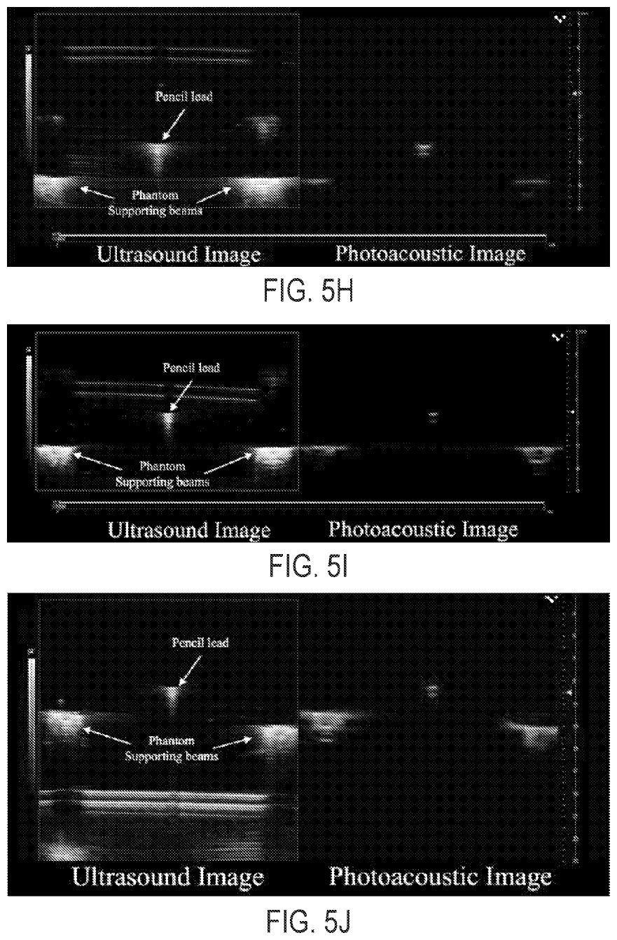

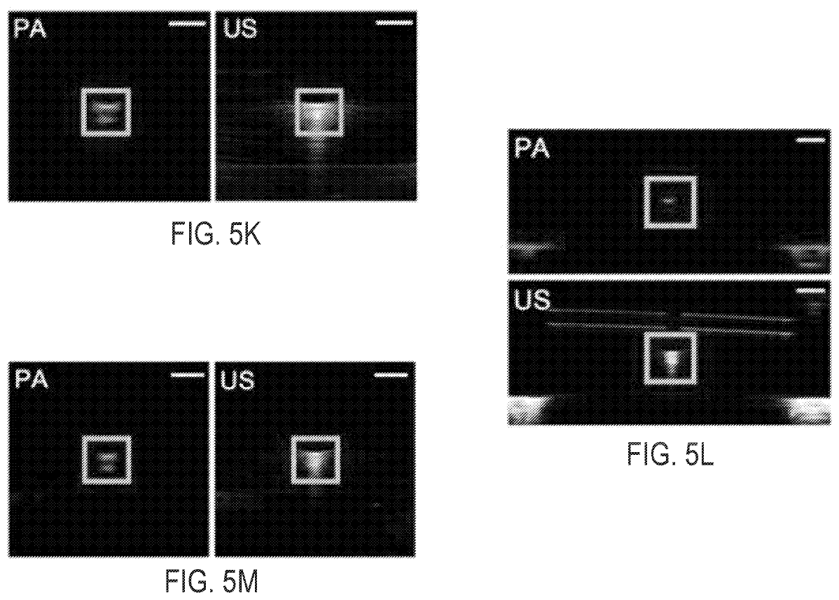

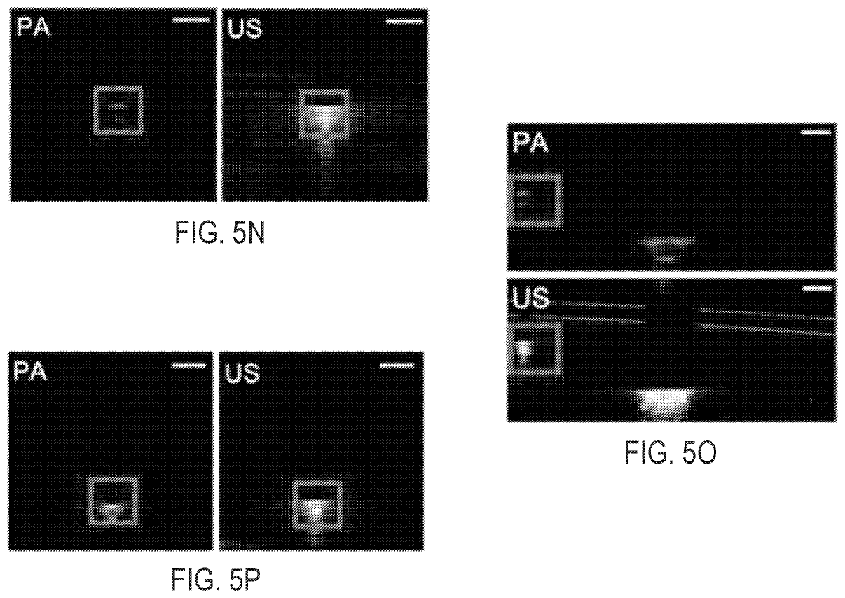

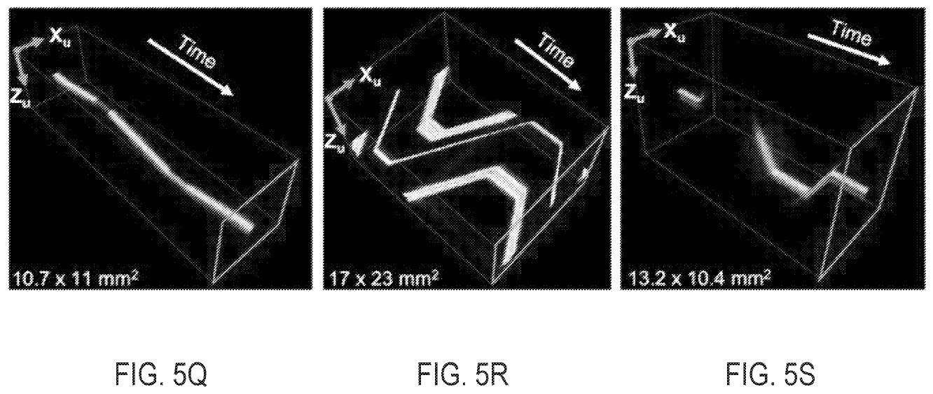

We characterized the orientation of the T265 camera with respect to the imaging axes of the Vevo LAZR-X system (schematically represented in FIG. 5A). It is critical to gauge the axes transformation between different systems (i.e., between the real world, camera and the imaging frame axes) to enable accurate 3D reconstruction. To avoid any uncertainty in the scan direction required for reconstruction, three main co-ordinates were chosen to describe motion in this study. The ‘front and back’, ‘left and right’ and ‘up and down’ motions were defined as X_{W}, Y_{W} and Z_{W} respectively. The world frame (i.e., the real world) acted as the ground reference for the other two co-ordinate systems as shown in FIG. 5A. The T265 co-ordinate system was defined as X_{C} (long axis of the camera), Y_{C} (short axis) and Z_{C} (height) with camera center as the origin. The orientation of the camera and world frame axes are the same. The 2D USPA image axes are defined as X_{U} (width of the frame) and Z_{U} (depth of the frame) with origin at the first pixel. The elevational direction or the axis on which the transducer is scanned for 3D USPA imaging is defined as Y_{U} axis. In this study, all the axes are color coded where green represented Z axis (up and down in all co-ordinates), red represented Y axis and blue represented the X axis respectively.

To characterize if the T265 camera can record the linear movements of the transducer in the three primary world axes, i.e., along X_{W}, Y_{W} and Z_{W} directions, a simple zig-zag motion was programmed on the linear stage to which the transducer was attached. The schematic representation of the phantom with a 0.7 mm lead (black) in between two supporting beams (grey) was used for USPA imaging as shown in FIGS. 5B-5D, where the black arrow depicts the direction of the integrated probe motion and yellow dashed line was the scan length. FIGS. 5E-5G exhibit the pose data obtained from the T265 camera when the integrated probe was moved in X_{C}, Y_{C} and Z_{C} direction respectively. Minimal motion was recorded by the camera for axes in which the integrated probe did not move. USPA images of the pencil lead (point source) were continuously acquired during the motion of the integrated probe. Snapshots of the acquired USPA images are displayed in FIG. 5H, FIG. 5I, and FIG. 5J, representing the motion in the X_{C} (forward and backward), Y_{C} (left and right) and Z_{C} (up and down) axes respectively.

We can also notice from FIGS. 5H-5J that the motion recorded by the T265 camera was also observed in the USPA images. FIGS. 5K-5M exhibit 2D PA and US frames from the original position (as pointed by the orange arrow in FIGS. 5E-5G with the lead cross-section highlighted in orange box. Similarly, FIGS. 5N-5P exhibit 2D PA and US frames at the timepoint specified with magenta arrow in FIGS. 5E-5G, with the lead cross-section highlighted in magenta box. For example, in FIG. 5G, the integrated probe was moved down by 3 mm, moved back up by a total of 6 mm and moved down by 3 mm for it to return to its original position. The USPA images in FIGS. 5K-5P corroborate with the pose data where the lead cross-section has moved down i.e., the magenta box is lower than the orange box. It is clear that the same motion is recorded by the T265 camera in the Z_{C} axis (FIG. 5G, (green line)) while no motion was recorded in the X_{C} (FIG. 5G, (blue line)) and Y_{C} axes (FIG. 5G, (red line)), as expected. Similar motion pattern of “no motion, move certain distance at constant speed, no motion, move back double the distance at constant speed, no motion, and return to original position” was observed for the other two primary axes. FIGS. 5Q-5S display the 2D USPA images as a 3D image with time as the third axis (represented by the white arrow). FIG. 5Q is the stack of images acquired when probe moved along X_{C} axis, i.e., along the pencil lead. Hence, no lateral motion is seen. FIG. 5R represents the stack of images displayed as 3D image when the probe moved along the Y_{C} axis. Clearly, the zig-zag motion along the X_{U} axis of the images is seen. As noted in FIG. 5A, motion in the Y_{C} axis translates to movement in the X_{U} axis. FIG. 5S displays the stack of USPA images acquired when the probe was moved up-down in Z_{C} axis. The fibers attached to the ultrasound probe focus light at 10 mm from the transducer surface, therefore when the pencil lead was too close to the transducer, it was out-of-light focus making the PA signal very weak or absent. However, as FIG. 5J indicates, the pencil lead can be clearly seen in US images. The pencil lead is not seen in photoacoustic image when it is out of laser focus.

### 3.2 Evaluating Accuracy, Repeatability and Minimum Incremental Detectable Distance of the T265 Camera

We established the jitter noise of the T265 camera by collecting the pose data when it is not in motion. The unprocessed pose data (non-smoothened) collected over 3 separate days and 3-5 different experiments on each day had approximately a standard deviation of 0.133 mm, 0.199 mm and 0.158 mm in the X_{C}, Y_{C} and Z_{C} directions respectively. As shown FIG. 4A, the camera was placed facing the ceiling while obtaining the data. If the camera was placed facing a dynamic environment where the participants move around in the room while the camera remained stationary, the standard deviation of the jitter noise was 7.34% and 12.79% higher in the X_{C} and Y_{C} directions and 16.09% lower in the Z_{C} direction. The X_{C} (front and back) and Y_{C} (left and right) axes are the predominant scanning directions for USPA imaging and hence the camera was configured to face the ceiling due to lower jitter in these directions. The movement in the Z_{C} (up and down) direction is not a predominant scanning direction because it will cause the transducer to move away from the object and create a loss of contact (i.e., acoustic mismatch due to air in between) between the transducer and the object being imaged.

We evaluated the positional accuracy of the camera which is defined as the measure of the error in distance indicated by the camera vs the actual distanced moved, where

$$Accuracy ( \% ) = 100 - error ( \% ) \qquad ( 1 )$$

$$Error ( \% ) = 100 * \frac{( distance estimated - acutal distance )}{actual distance} \qquad ( 2 )$$

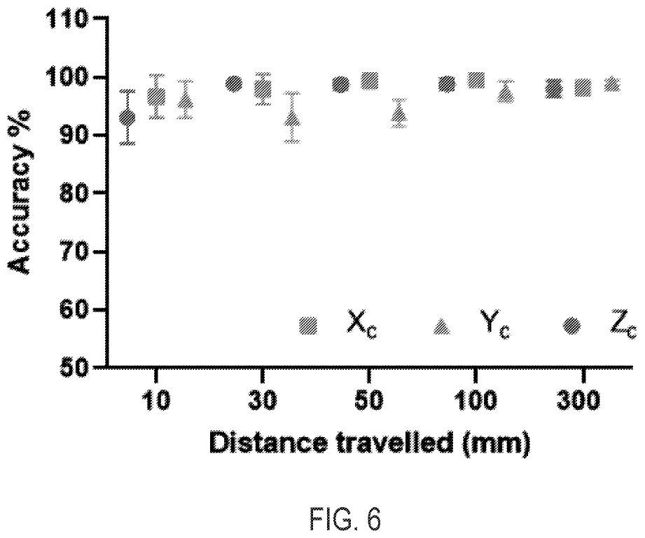

For distances ranging 10 mm to 300 mm and camera moving at speeds 1 mm/s-10 mm/s, the average accuracy of the camera for all distances is 98.33%, 95.94% and 97.49% along the X_{C}, Y_{C}, and Z_{C} axes respectively (Table S1). These accuracies are in the similar range as those previously reported with T265 camera in autonomous robotic applications for larger travelling distances. In addition, the accuracy (%) is lower for shorter distances travelled. Given that T265 camera was not previously used for photoacoustic imaging applications or smaller travel distances, this is the first report to provide the accuracy for distances in the centimeter range or lower. Specifically in these studies, accuracy was 96.67% for 10 mm travel distance but accuracy was 98.18% for 300 mm travel distance, along the X_{C} axis. FIG. 6 and Table S1 clearly shows the accuracy (%) is higher with increased travel distance in all the three axes directions. Furthermore, motion along the Y_{C} axis (short axis of the camera) had the lowest accuracy as was also previously observed in large scale applications.

In addition to the accuracy, the positional repeatability of the camera was calculated, where repeatability is defined as the extent to which successive attempts to move to a specific location vary in position i.e., the error in the pose data in reporting the position time after time. The position reported by the camera pose data was compared to the position set on the linear stage to calculate the error. Standard deviation of the error is reported as the repeatability of the camera. It has to be noted that the linear stage inherently has an accuracy of 20 μm and repeatability error of ~3 μm. Ignoring the impact of this error on the results, over several runs, for several distances, the average repeatability to be 210.1 μm, 79.4 μm and 457.4 μm along X_{C}, Y_{C} and Z_{C} axes respectively (Table 1).

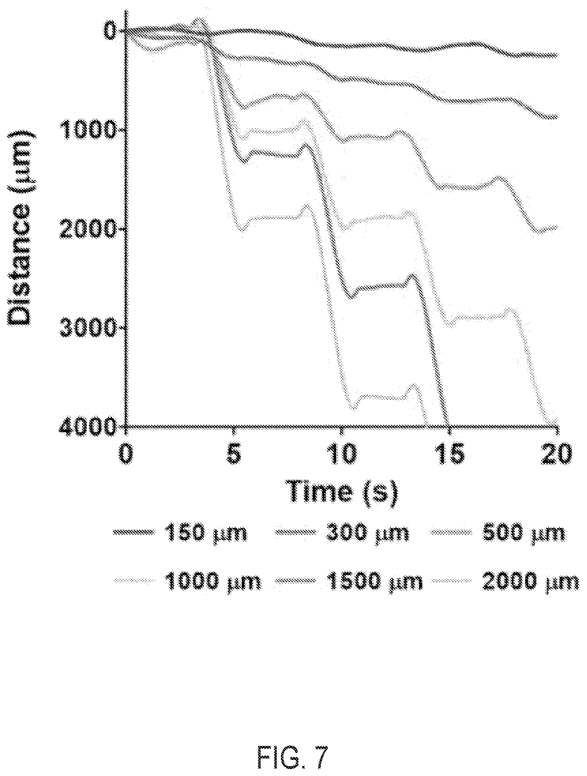

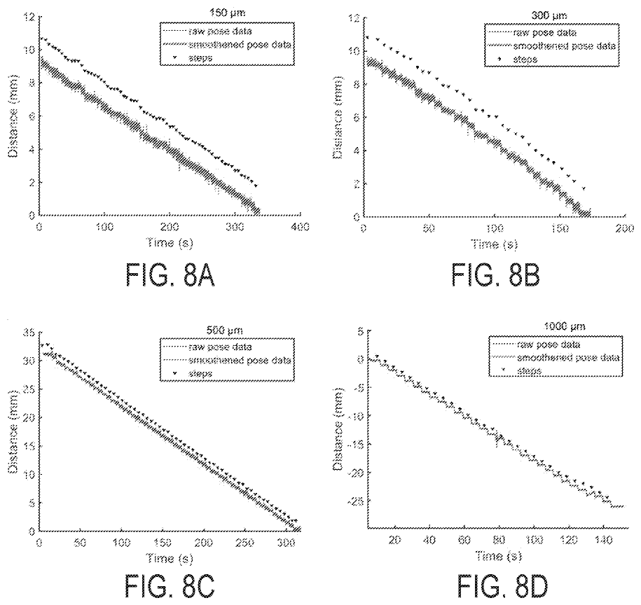

The next study involved evaluation of the minimum distance reliably tracked by the T265 camera. FIG. 7 shows the smoothened pose data for various step-sizes. The minimum step-size reliably differentiatable from the background jitter in the pose data was found to be 500 μm as shown in FIG. 7, where the steps were clearly identified. Specifically, the accuracy for 500 μm, 1000 μm, 1500 μm, and 2000 μm step sizes was 90.46%, 91.52%, 91.32% and 90.51% respectively and the repeatability was 120.92, 159.84, 131.80 and 135.55 μm respectively. The findpeaks command on MATLAB also identified the accurate number of step-sizes above 500 μm (Table 2). For step-sizes less than 500 μm, differentiating and computing number of steps from the pose data was not reliable and did not match the number of USPA frames acquired (Table 2). In other words, the accuracy of the camera in detecting these small step sizes of 150 μm and 300 μm was less than 50% and these step-sizes were in the repeatability error range mentioned above. FIG. 8A-8D the raw pose data with an overlay of smoothened pose data acquired in FIG. 7.

TABLE 1
Accuracy and repeatability of the camera pose data compared
to distance programmed on a linear stage for various
distances travelled at 1, 5 and 10 mm/s speed along all
three axes (n = 3 measurements for each row).

| | | | | | |
|---|---|---|---|---|---|
| | | Distance | | | |
| Distance | | measured | | | |
| on linear | Speed | on camera | Error | Accuracy | Repeatability |
| stage (mm) | (mm/s) | (mm) | (mm) | (%) | (mm) |

X [symbol] Axis

| | | | | | |
|---|---|---|---|---|---|
| 10 | 1 | 9.9479 | 0.052 | 98.21 | 0.1162 |
| | 5 | 9.9624 | 0.037 | 97.59 | |
| | 10 | 9.7542 | 0.245 | 94.21 | |
| 30 | 1 | 29.4256 | 0.574 | 97.89 | 0.1020 |
| | 5 | 29.6295 | 0.370 | 98.26 | |
| | 10 | 29.5344 | 0.465 | 97.57 | |
| 50 | 1 | 49.7599 | 0.240 | 99.51 | 0.1018 |
| | 5 | 49.8755 | 0.124 | 99.34 | |
| | 10 | 49.9627 | 0.037 | 99.26 | |
| 100 | 1 | 99.9834 | 0.016 | 99.56 | 0.1513 |
| | 5 | 99.7234 | 0.276 | 99.49 | |
| | 10 | 99.9875 | 0.012 | 99.65 | |
| 300 | 1 | 295.2327 | 4.767 | 98.41 | 0.5794 |
| | 5 | 294.1789 | 5.821 | 98.05 | |
| | 10 | 294.2882 | 5.711 | 98.09 | |

| | |
|---|---|
| Average accuracy (%) | 98.33% |

| | |
|---|---|
| Average repeatability (mm) | 0.2101 |

Y [symbol] Axis

| | | | | | |
|---|---|---|---|---|---|
| 10 | 1 | 10.2097 | 0.209 | 95.50 | 0.0217 |
| | 5 | 10.2349 | 0.234 | 96.84 | |
| | 10 | 10.2530 | 0.253 | 96.21 | |
| 30 | 1 | 32.0486 | 2.048 | 93.17 | 0.0691 |
| | 5 | 32.1574 | 2.157 | 92.80 | |
| | 10 | 32.0291 | 2.029 | 93.23 | |
| 50 | 1 | 53.1066 | 3.106 | 93.78 | 0.0533 |
| | 5 | 53.0007 | 3.000 | 93.99 | |
| | 10 | 53.0630 | 3.063 | 93.87 | |
| 100 | 1 | 102.4583 | 2.458 | 97.54 | 0.1293 |
| | 5 | 102.5530 | 2.553 | 97.44 | |
| | 10 | 102.2972 | 2.297 | 97.72 | |
| 300 | 1 | 302.9770 | 2.977 | 99.00 | 0.1241 |
| | 5 | 303.0416 | 3.041 | 98.98 | |
| | 10 | 302.8018 | 2.801 | 99.06 | |

| | |
|---|---|
| Average accuracy (%) | 95.94% |

| | |
|---|---|
| Average repeatability (mm) | 0.0795 |

Z [symbol] Axis

| | | | | | |
|---|---|---|---|---|---|
| 10 | 1 | 10.7809 | 0.780 | 92.19 | 0.1120 |
| | 5 | 10.5673 | 0.567 | 94.32 | |
| | 10 | 10.7323 | 0.732 | 92.67 | |
| 30 | 1 | 29.8633 | 0.136 | 98.56 | 0.1062 |
| | 5 | 29.8807 | 0.119 | 99.08 | |
| | 10 | 30.0554 | 0.055 | 98.92 | |
| 50 | 1 | 49.2158 | 0.784 | 98.43 | 0.1343 |
| | 5 | 49.4391 | 0.560 | 98.87 | |
| | 10 | 49.4569 | 0.543 | 98.86 | |
| 100 | 1 | 98.7965 | 1.203 | 98.79 | 0.1332 |
| | 5 | 98.6858 | 1.314 | 98.68 | |
| | 10 | 98.9510 | 1.049 | 98.95 | |
| 300 | 1 | 292.7644 | 7.235 | 97.58 | 1.8014 |
| | 5 | 293.2443 | 6.755 | 97.74 | |
| | 10 | 296.0966 | 3.903 | 98.69 | |

| | |
|---|---|
| Average accuracy (%) | 97.49% |

| | |
|---|---|
| Average repeatability (mm) | 0.4574 |

[symbol]

indicates data missing or illegible when filed

TABLE 2
Comparison of step-sizes recorded on the T265 camera with
the number of USPA frames acquired during linear move-
stop-acquire image-repeat motion in Yc direction.

| Step | No of USPA | | No of | | |
|---|---|---|---|---|---|
| size | frames (total | | steps | Accuracy | Repeatability |
| (μm) | scan distance) | | from IMU | (%) | (μm) |
| 150 | 66 | (3 cm) | 63 | <50% | N/A |
| 300 | 33 | (3 cm) | 35 | | |
| 500 | 61 | (10 cm) | 61 | 90.46% | 120.92 |
| 1000 | 30 | (10 cm) | 30 | 91.52% | 159.84 |
| 1500 | 20 | (10 cm) | 20 | 91.32% | 131.80 |
| 2000 | 15 | (10 cm) | 15 | 90.51% | 135.55 |

3.3 Establishing the Maximum Speed that can be Used for Linear Motion of Integrated Probe with 20 Hz Nanoseconds Pulsed Laser

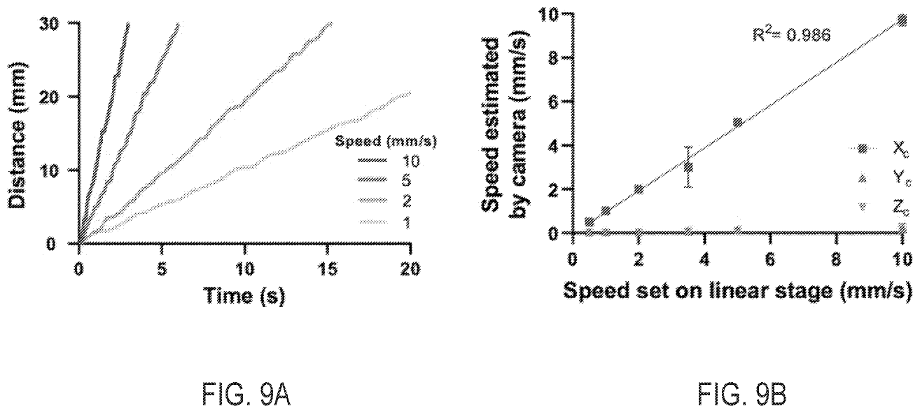

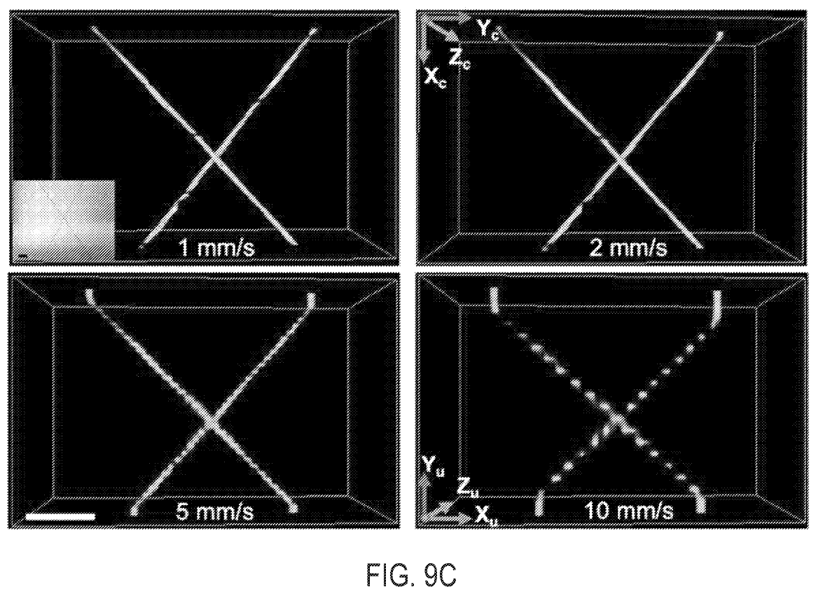

In systems such as the FujiFILM Vevo LAZR-X used in this study, the frame acquisition rate is about 5-20 Hz. Low frame rates accompanied with high-speed translation motion can lead to low sampling of the object being imaged and therefore an erroneous 3D reconstruction. Imaging systems such as the Vevo LAZR-X system utilize a “move-stop-acquire image-repeat” scanning methodology with the linear translational stage. However, such a scenario with pre-determined step sizes is not possible with free hand imaging. To characterize the maximum speed at which the integrated USPA probe can be used for reliable 3D reconstruction of the object without loss of data, the integrated probe was attached to a linear stage that moved at constant speed. The speed ranged from 0.5 mm/s to 10 mm/s for a fixed travel distance of 30 mm (FIG. 9A). A linear correlation analysis was performed on the speeds set on the linear stage and speed calculated from the T265 camera's pose data and R2=0.986 was observed (FIG. 9B). It can also be noted that speed across other axis was zero (red and green data points in FIG. 9B). USPA images of hair phantom were also acquired while translating the integrated probe at various speeds. A higher number of USPA frames at slower speeds were obtained than at higher speeds, as expected. As seen in FIG. 9C, 3D reconstruction of USPA images at 10 mm/s was missing significant structural information, such as the intersection of the two hair strands. At speeds 5 mm/s or lower, the motion compensated reconstructions were similar to the actual phantom. As USPA images were acquired with 20 Hz frame rate, the minimum distance traversed between two adjacent frames was 250 μm for a 5 mm/s travel speed. Though the distance between adjacent frames is in the range of the elevational resolution of the transducer (~300 μm), it does not satisfy the Nyquist criterion, but can be used for 3D reconstruction with interpolation between frames for qualitative 3D representation. A travel speed of 3 mm/s that generates ~150 μm distance between frames or lower speeds will be required for accurate 3D reconstructions. With availability of pulsed lasers that operate at high pulse repetition frequency, several frames can be acquired satisfying the Nyquist criterion and providing accurate 3D reconstruction of the object being imaged.

### 3.4 Quantitative Comparison of Scanning Speeds of Various Users Using the Integrated Probe for Reliable 3D Reconstruction

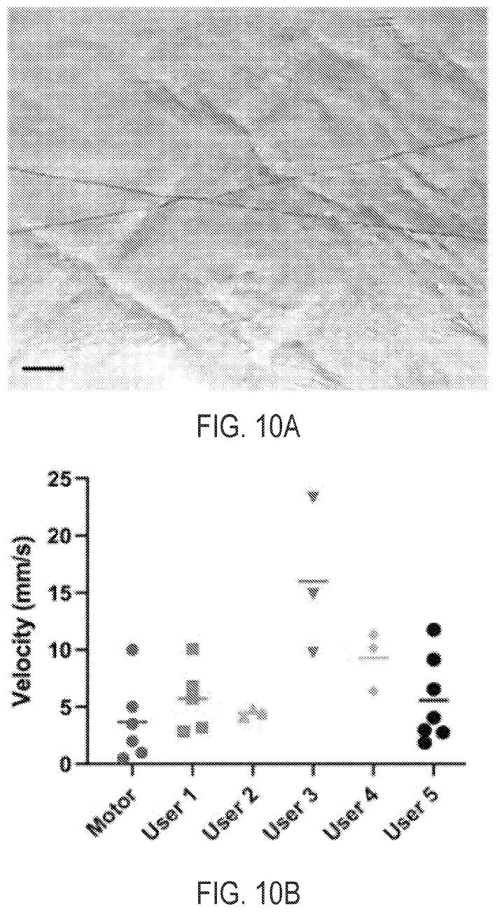

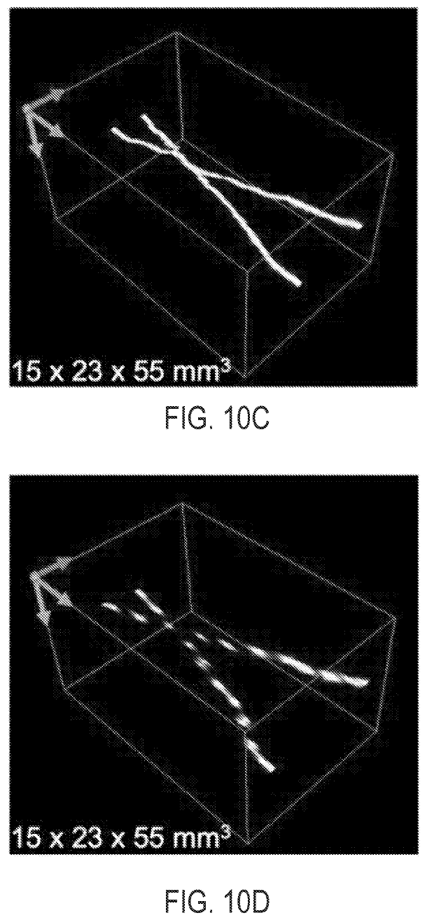

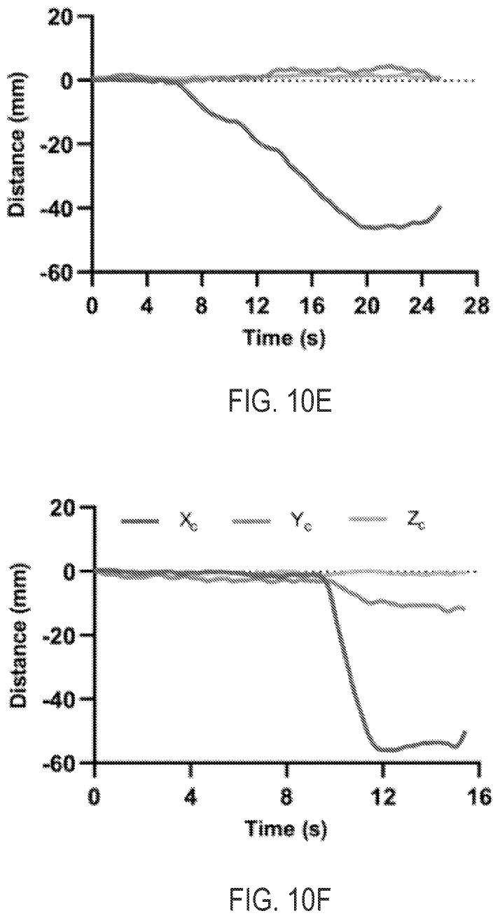

Our next step was to evaluate the potential of 3D reconstruction of the gelatin hair phantom (FIG. 10A) when imaged by various users that were not pre-trained on holding the integrated probe but were familiar with USPA imaging. The users were instructed to perform multiple scans while looking at the near real time USPA images on the Vevo LAZR-X screen. Scan speeds of all the users calculated from the T265 camera pose data are reported in FIG. 10B. As can be noted there were inter- and intra-scanning speed differences between users. User 3 has the highest average scan speed whereas the User 2 has the most consistent scan speed. Similar to 3D reconstructions for scan performed by a motor at various speeds (section 3.3 (above)), users who scanned at lower speeds (example User 2) were able to capture higher number of USPA image frames while users who moved the integrated probe at higher speeds had low number of USPA frames (example User 3) as expected. Obtaining high number of USPA image frames (low speed while moving the integrated probe) produced a better 3D reconstruction of the phantom than that of 3D reconstruction from users who scanned at higher speed. As shown in FIGS. 10C-10D, when imaged at lower speed by User 2 (~3.5 mm/s) and higher speed by User 3 (14 mm/s) respectively, the 3D reconstruction was better in the former case. Corresponding translational pose data acquired by the T265 camera for the handheld scans shown in FIGS. 10E-10F. Clearly, the slope (speed) of the X_{C} pose data is steeper for the higher speed scan. It was observed that the users were able to maintain constant speed for the duration of the scan for lengths 10-15 cm. If the user cannot maintain a constant scan speed for longer scans lengths, no limitations are anticipated for the 3D reconstruction as the actual position and orientation data is used and not the speed at a particular time point. Overall, for a USPA imaging system operating at 20 Hz frame rate, a scan speed of 5 mm/s or less would be optimal.

### 3.5 Comparison of the Range of Motion of Linear Stage VS Handheld Imaging Probe

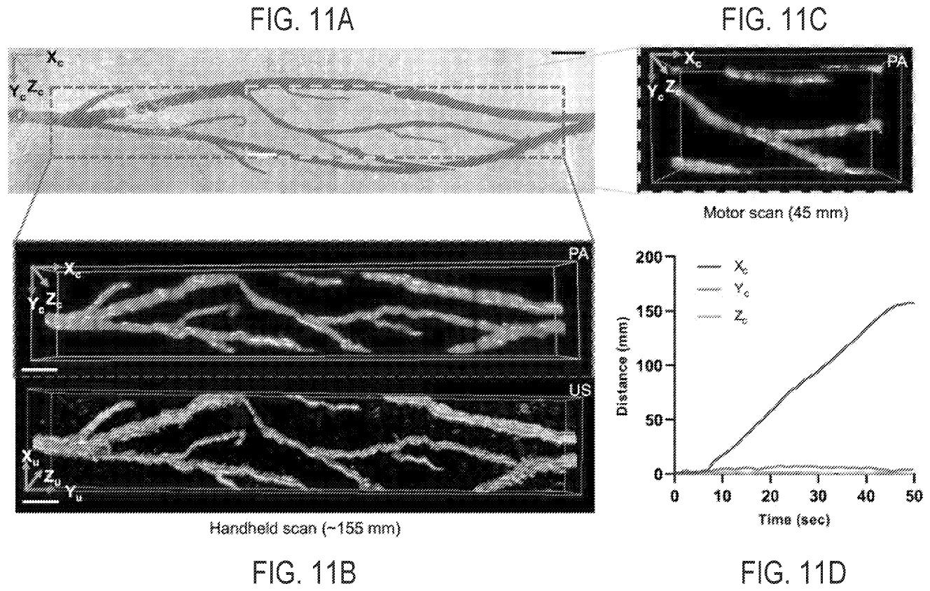

The range of motion that can be achieved with linear motor and the handheld probe are compared in FIG. 11A phantom of approximately 160 mm length was used for this experiment (FIG. 11A). FIG. 11B-11C depict the 3D PA and US handheld data, and linear stage. Due to the limited range on the linear stage, only 45 mm scan length was possible. However, with handheld scan, the whole length of the phantom was able to be imaged, as shown in FIG. 11B. Clearly, a high visual correlation between the phantom picture and the handheld motion compensated 3D reconstruction can be observed. Although, linear stages with larger range can be purchased, they can be bulky and non-portable. In certain cases, multiple linear stages could be required to capture the whole phantom in 3D, which can significantly increase the imaging time and 3D reconstruction complexity. With the integrated handheld probe, the entire phantom was able to be imaged in a single scan. Due to this feature, the integrated probe has high potential to image large scan areas making it one of the main advantages of this probe.

3.6 Volume Estimation from the Images Acquired with the Integrated T265 and USPA Probe

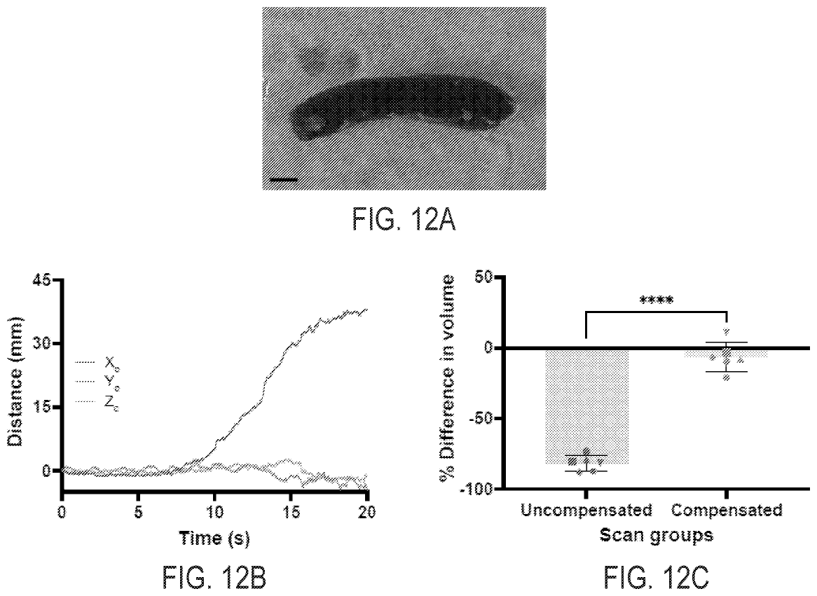

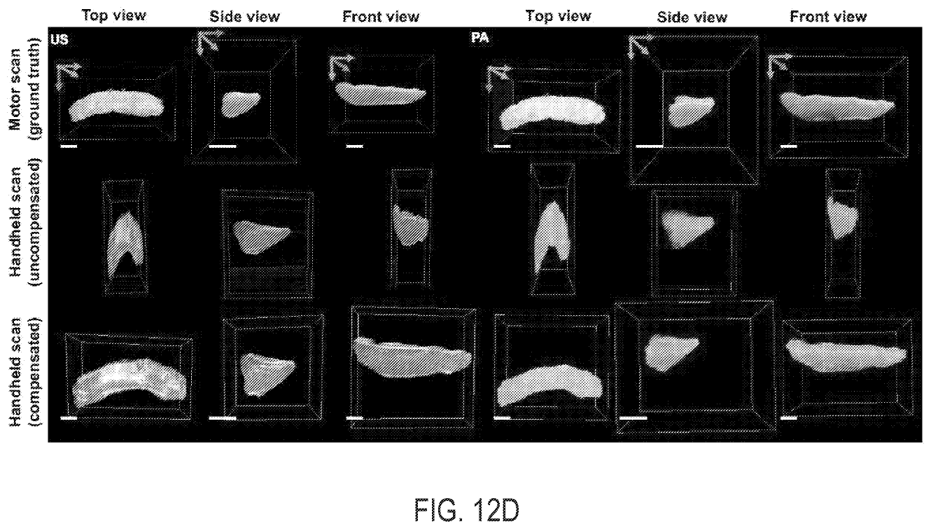

Post characterization of the 3D handheld integrated USPA imaging probe, its ability to estimate the true volume of a tissue with the pose data acquired from the T265 camera was explored. The handheld reconstructed volume was compared to the volume calculated from linear stage scan. The stack of 2D images during the handheld scans are referred as ‘motion uncompensated’ data, whereas the 3D reconstruction of these 2D interpolated images using the pose data is referred as ‘motion compensated’ data. A rat spleen ex vivo embedded in a gelatin phantom (FIG. 12A) was imaged using the integrated probe. FIGS. 12B-12C summarize the volume analysis on reconstruction of freehand scans (6 different users) on the rat spleen phantom. The ultrasound and photoacoustic 3D reconstructions of the spleen in three different views (top, side and front) was displayed in FIG. 12D. The top view of the spleen shown in FIG. 12D matches with the ground truth and motion compensated 3D images but not the uncompensated handheld scanned image. The uncompensated handheld images (FIG. 12D middle panel) are compressed in the Y_{U} axis due to the unavailability of the scan length. This suggests that uncompensated 3D reconstruction underestimated the volume of the spleen. Representative pose data from a handheld scan of the spleen phantom is shown in FIG. 12B. There was no motion up until 9 seconds after the start of the acquisition. After 9 seconds, there is translation in the X_{C} axis (Y_{U} in the USPA image frame). Upon motion compensation using the pose data from the T265 camera (FIG. 12B), a 3D reconstruction of a freehand scan was accomplished, which is now structurally similar to the ground truth. Volume of the spleen was estimated from the manually segmented USPA images for uncompensated and motion-compensated 3D data using MATLAB segmentation toolkit. As mentioned previously, here the volume estimated was assumed from the linear translation stage as the true volume. Percentage difference in volume of the spleen from the ground truth is plotted in FIG. 12C for motion compensated and uncompensated images respectively for data obtained by six different users. Clearly the difference in volume between the ground truth and handheld scan is averaging around zero as expected (FIG. 12C, orange bar). A simple t-test produced p-values <0.0001, indicating the percentage difference in volume of uncompensated and compensated volumes calculated for all freehand scans is significantly different.

Very recently Jiang et al. have used a GPS based system for 3D photoacoustic imaging using G4 system from Polhemus Inc. The G4 system offers similar features like the T265 camera, i.e., it is portable, scalable and compact (similar size) but the major difference is that the G4 system is a 3-piece electromagnetic tracking system while the T265 camera combines inertial tracking with Vi-SLAM algorithms in one system combinedly referred to as Visual odometry system. Electromagnetic tracking units may experience interference when operating in the vicinity of devices that produce magnetic fields and metal objects present in the rooms can also disrupt the magnetic fields. Jiang et al. have taken additional precautions to avoid presence of magnetic distortion by specifically placing the sensor 8 cm behind the middle line of linear array probe. While the T265 camera is not impacted by electromagnetic distortions, studies have shown that the performance of the T265 camera is impacted by bright light such as sunlight in outdoor environments. However, such bright lights are unusual in a laboratory or a clinical environment, making the visual odometry based freehand USPA imaging a viable technique for 3D visualization of tissues as demonstrated by the results.

### 4. Conclusions

Thus, a low-cost, adaptable, and system independent freehand 3D USPA imaging probe was developed. The handheld probe was a combination of the ultrasound transducer to acquire USPA signals, fiber optics to deliver laser pulses and the Intel T265 camera which consists of two fisheye cameras and an IMU sensor for tracking the probe position. Intel® RealSense™ was primarily used in robotics for localization, where the range of motion is in meters. This was the first time where such cameras are utilized for photoacoustic imaging. While similar IMU based systems were previously used for ultrasound imaging and have been extensively reviewed elsewhere, here the use of visual odometry for the first time for combined ultrasound and photoacoustic imaging was presented. The camera facing the ceiling of the room provides a viable option to avoid such distortions due to dynamic environment and real-world clinical rooms and imaging suites have ceilings with railings and other patterns (false ceiling) that can act as fiducial landmarks. If rooms have ceilings that are devoid of patterns or fiducial markers, taping printed patterns on the ceiling could resolve the issue. However, the ergonomics and functionality of the integrated probes in different environments with different ceiling patterns needs further investigation and are out of the scope of the current work that is focused on demonstrating the feasibility of using visual odometry for combined 3D USPA imaging.

The Intel® RealSense™ T265 camera was chosen primarily due to its low cost and relatively better performance than other readily available odometers. The T265 camera, being a single unit system, can be attached to any transducer operating at lower frequencies than that used in this study (20 MHz) as the calculated accuracy, jitter noise, repeatability and minimum incremental distance calculated for the current camera are on the order of lateral and elevational resolution of the transducer used in this study. It is anticipated that sensors with micrometer range accuracy and precision will be readily available and can be integrated with such handheld systems while being economical, accurate, portable, and less bulky. The T265 camera provided 6 DOF pose information, i.e., both translational and rotational information of the transducer is provided. In this study the salient features of utilizing T265 tracking camera for linear translation motion was demonstrated and optimized the scanning speed for handheld imaging.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The various illustrative logics, logical blocks, modules, circuits and algorithm processes described in connection with the implementations disclosed herein may be implemented as electronic hardware, computer software, or combinations of both.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor or any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by or to control the operation of data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that can be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Similarly, while operations are depicted in the drawings in a particular order, this should not be understood as requiring that such operations be performed in the particular order shown or in sequential order, or that all illustrated operations be performed to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

1. An apparatus for three-dimensional (3D) image reconstruction, the apparatus comprising:
a housing configured for freehand movement within a 3D reference frame defined by an X_{W}-axis, Y_{W}-axis, and Z_{W}-axis, the housing comprising:
a laser source configured to transmit an electromagnetic (EM) wave into a sample to produce a photoacoustic effect therein;
an ultrasound probe configured to generate scans of the sample;
a visual odometer configured to track movement of the ultrasound probe in the 3D reference frame to generate odometer data; and
a processor in communication with the ultrasound probe and the visual odometer, configured to:
receive the scans and odometer data, wherein the scans and odometer data each include timestamps,
synchronize the scans and odometer data, and
construct a 3D image of the scans based on the synchronization.

2. The apparatus of claim 1, wherein the ultrasound probe includes one or more one-dimensional (1D) or two-dimensional (2D) array transducers.

3. The apparatus of claim 1, wherein the visual odometer includes at least one optical sensor and an inertial measurement unit (IMU).

4. The apparatus of claim 3, wherein the at least one optical sensor includes a visible light camera, infrared camera, or light detecting and ranging (LiDAR) camera.

5. The apparatus of claim 3, wherein the at least one optical sensor includes a fisheye camera.

6. The apparatus of claim 3, wherein the optical sensor is configured to be directed toward the sample or away from the sample.

7. The apparatus of claim 3, wherein the IMU includes a tri-axial gyroscope, a tri-axial accelerometer, and optionally a tri-axial magnetometer.

8. The apparatus of claim 7, wherein the processor is further configured to fuse data acquired by the optical sensor, gyroscope, accelerometer, and optional magnetometer into the output data including a six degrees of freedom (6 DOF) pose and an orientation of the optical sensor.

9. The apparatus of claim 8, wherein the processor is further configured to identify a motion of a feature in the scans.

10. The apparatus of claim 9, wherein the feature includes a speckle pattern.

11. The apparatus of claim 8, wherein the processor is further configured to trim the odometer data to match the scans.

12. The apparatus of claim 11, wherein the processor is further configured to apply a smoothing filter to the output data to generate smooth data with reduced noise.

13. The apparatus of claim 12, wherein the processor is further configured to divide the smoothed output data by a spatial resolution of the ultrasound probe in each of the x-axis, y-axis, and z-axis to determine a pixel shift.

14. The apparatus of claim 13, wherein the processor is further configured to up-sample the scans to match a frame rate of the optical sensor.

15. The apparatus of claim 14, wherein the processor is further configured to spatially align the scans in a 3D space based on a transformation, wherein the transformation includes the pixel shift.

16. A method for three-dimensional (3D) image reconstruction, the method comprising:
receiving, using a processor, photoacoustic ultrasound scans from an ultrasound probe within a housing configured for freehand movement within a 3D reference frame defined by an X_{W}-axis, Y_{W}-axis, and Z_{W}-axis;
receiving, using the processor, odometer data from a visual odometer within the housing configured to track movement of the ultrasound probe in the 3D reference frame;
synchronizing the scans and the odometer data based on timestamps associated with each of the scans and the odometer data;
constructing a 3D image of the scans based on the synchronization.

17. The method of claim 16, wherein the ultrasound probe includes one or more one-dimensional (1D) or two-dimensional (2D) array transducers.

18. The method of claim 16, wherein the visual odometer includes at least one optical sensor and an inertial measurement unit (IMU).

19. The method of claim 18, wherein the at least one optical sensor includes a visible light camera, infrared camera, or light detecting and ranging (LiDAR) camera.

20. The method of claim 18, wherein the at least one optical sensor includes a fisheye camera.

21. The method of claim 18, wherein the optical sensor is configured to be directed toward the sample or away from the sample.

22. The method of claim 18, wherein the IMU includes a tri-axial gyroscope, a tri-axial accelerometer, and optionally a tri-axial magnetometer.

23. The method of claim 22, further comprising, using the processor, fusing data acquired by the optical sensor, gyroscope, accelerometer, and optional magnetometer into the output data including a six degrees of freedom (6 DOF) pose and an orientation of the optical sensor.

24. The method of claim 23, further comprising, using the processor, identifying a translational motion of a feature in the scans.

25. The method of claim 24, wherein the feature includes a speckle pattern.

26. The method of claim 23, further comprising, using the processor, trimming the odometer data to match the scans.

27. The method of claim 26, further comprising, using the processor, applying a smoothing filter to the odometer data to generate smooth data with reduced noise.

28. The method of claim 27, further comprising, using the processor, dividing the smoothed odometer data by a spatial resolution of the ultrasound probe in each of the x-axis, y-axis, and z-axis to determine a pixel shift.

29. The method of claim 28, further comprising, using the processor, up-sampling the scans to match a frame rate of the optical sensor.

30. The method of claim 29, further comprising, using the processor, spatially aligning the scans in a 3D space based on a transformation, wherein the transformation includes the pixel shift.
